(19) United States
(12) Patent Application Publication
(10) Pub. No.: US 2004/0248816 A1
(43) Pub. Date: Dec. 9, 2004

(54) Treatment of migraine

(75) Inventors: Henri Doods, Warthausen (DE); Klaus Rudolf, Warthausen (DE); Wolfgang Eberlein, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/811,515

(22) Filed: Mar. 29, 2004

Related U.S. Application Data
(60) Provisional application No. 60/463,069, filed on Apr. 15, 2003.

(30) Foreign Application Priority Data
Apr. 1, 2003 (DE) DE10314617.2

(51) Int. Cl. A61K 38/04; A61K 31/517

(52) U.S. Cl. 514/19; 514/252.17

(57) ABSTRACT

The present invention relates to a process for the treatment or prevention of headache, migraine and cluster headaches, this process comprising the joint administration of a therapeutically effective amount of the hydrochloride of the active substance base 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine (A) and of a therapeutically effective amount of the anti-migraine substance sumatriptan or a physiologically acceptable salt thereof, and the corresponding pharmaceutical compositions and the preparation thereof.

This application claims benefit from U.S. Provisional Application Ser. No. 60/463,069, filed Apr. 15, 2003, which application is incorporated herein by reference in its entirety.

### BACKGROUND OF THE INVENTION

Migraine is one of the most common neurological disorders and comprises periodic attacks of headache and nausea and a variety of other symptoms. Although considerable progress has been made in the past, the pathophysiology of migraine is far from understood. A number of observations have pointed to the involvement of the “calcitonin gene related peptide” (CGRP).

Migraine headaches involve the activation of the trigeminal system and the dilation of cranial blood vessels. CGRP is located in the neurons in trigeminal ganglia, and the CGRP levels are raised during a migraine attack. These elevated CGRP levels cause vasodilatation and are thus presumably responsible for the headache. It is therefore conceivable that inhibiting the dilation of the cranial blood vessels caused by CGRP might possibly give rise to a new treatment for migraine headaches.

Medicaments widely used for treating migraine are the so-called “triptans”, e.g. almotriptan, eletriptan, frovatriptan, naratriptan, rizatriptan, sumatriptan and zolmitriptan. These compounds derive their activity against migraine from their vasoconstrictor properties and presumably their inhibition of the release of the neuropeptide “calcitonin gene related peptide” (CGRP) (Ferrari, M. D., Saxena, P. R. (1995), 5-HT₁ receptors in migraine pathophysiology and treatment, Eur. J. Neurology, 2, 5-21; Johnson, K. W., Phebus, L. A., Cohen, M. L. (1998), Serotonin in migraine: Theories, animal models and emerging therapies, Progress in Drug Research, vol. 51, 220-244), assuming that the levels thereof are raised during a migraine attack (Edvinsson, L., Goadsby, P. J. (1994), Neuropeptides in migraine and cluster headache, Cephalgia, 14(5), 320-327). A completely new approach for the treatment of migraine is the use of CGRP antagonists (Doods, H., Hallermayer, G., Wu, D., Entzeroth, M., Rudolf, K., Engel, W., Eberlein, W. (2000), Pharmacological profile of BIBN4096BS, the first selective small molecule CGRP antagonist, Br. J. Pharmacol., 129, 420-423; Doods, H. (2001), Development of CGRP antagonists for the treatment of migraine, Curr. Opinion Investig. Drugs 2(9), 1261-1268).

International Patent Application PCT/EP9704862 (published as WO 98/11128) discloses modified amino acids with CGRP-antagonistic properties, the use thereof and processes for the preparation thereof as well as the use thereof for the preparation and purification of antibodies and as labelled compounds in RIA and ELISA assays and as diagnostic or analytical aids in neurotransmitter research. In view of their pharmacological properties the modified amino acids are therefore suitable for the acute and prophylactic treatment of headaches, particularly migraine and cluster headaches. Moreover, International Patent Application PCT/EP 0208993 (published as WO 03/015787) generally discloses the use of the CGRP antagonist 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine in conjunction with other antimigraine agents for the treatment of migraine.

### SUMMARY OF THE INVENTION

Surprisingly it was found that in a model assumed to predict the anti-migraine activities of pharmaceutical compositions, the combination of two pharmaceutical compositions with completely different modes of activity, namely a 5-HT_{1B/1D}-agonist and the hydrochloride of the CGRP antagonist 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine (A) disclosed in WO 98/11128 A1,

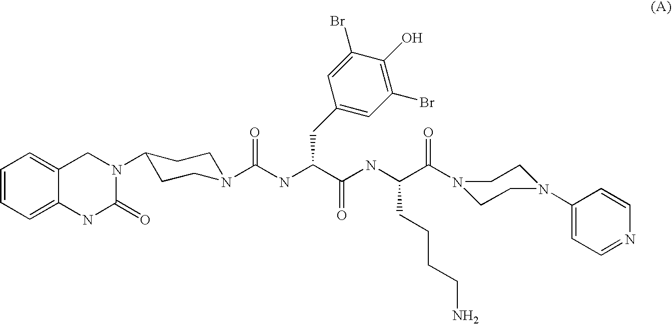

leads to a significantly improved activity compared with the activity of only one pharmaceutical composition.

### DETAILED DESCRIPTION OF THE INVENTION

According to a first aspect the present invention provides a process for the treatment or prevention of indications selected from the group comprising headaches, migraine and cluster headaches, this process comprising the simultaneous or sequential administration of a therapeutically effective amount of the hydrochloride of the active substance base 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine (A) and of a therapeutically effective amount of the 5-HT_{1B/1D}-agonist sumatriptan or one of the physiologically acceptable salts thereof to a person in need of such treatment.

The dosage for the combined anti-migraine agent sumatriptan or one of the physiologically acceptable salts thereof is roughly 1/10 of the lowest normally recommended dose to 1/1 of the normally recommended dose, preferably ⅓ to 1/1, by oral, nasal, inhalative, subcutaneous, rectal or intravenous route. The normally recommended dose for the combined anti-migraine agent sumatriptan is the dose prescribed in the Red List 2003, published by Editio Cantor of Aulendorf.

According to the invention, the hydrochloride of the active substance base (A) is administered by intravenous or subcutaneous route in a dosage of 0.0001 to 3 mg/kg of body weight or by oral, nasal, rectal or inhalative route in a dosage of 0.1 to 10 mg/kg of body weight, once, twice or three times a day, in conjunction with sumatriptan or a physiologically acceptable salt thereof, which may be administered

by oral route in a dosage of 0.14 to 1.5 mg/kg of body weight once, twice or three times a day or

by intravenous or subcutaneous route in a dosage of 0.009 to 0.1 mg/kg of body weight once or twice a day or

by rectal route in a dosage of 0.04 to 0.36 mg/kg of body weight once or twice a day or

by inhalation in a dosage of 0.057 to 0.57 mg/kg of body weight once or twice a day or

by nasal route in a dosage of 0.03 to 0.29 mg/kg of body weight once or twice a day.

In a preferred embodiment according to the invention the hydrochloride of the active substance base (A) may be administered by intravenous or subcutaneous route in a dosage of 0.0001 to 3 mg/kg of body weight or by oral, nasal, rectal route or by inhalation in a dosage of 0.1 to 10 mg/kg of body weight once, twice or three times a day, in conjunction with sumatriptan or a physiologically acceptable salt thereof, which may be administered

by oral route in a dosage of 0.48 to 1.5 mg/kg of body weight once, twice or three times a day or

by intravenous or subcutaneous route in a dosage of 0.03 to 0.1 mg/kg of body weight once or twice a day or

by rectal route in a dosage of 0.12 to 0.36 mg/kg of body weight once or twice a day or

by inhalation in a dosage of 0.19 to 0.57 mg/kg of body weight once or twice a day or

by nasal route in a dosage of 0.1 to 0.29 mg/kg of body weight once or twice a day.

In a second aspect the present invention claims a pharmaceutical composition for the treatment or prevention of headache, migraine or cluster headaches, which consists of a therapeutically effective amount of the hydrochloride of the active substance base 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine (A) and the anti-migraine agent sumatriptan or one of the physiologically acceptable salts thereof, as a combined preparation for simultaneous or sequential administration.

A pharmaceutical composition according to the invention may contain a single dosage unit of 0.1 to 10 mg of the hydrochloride of the active substance base (A) and a single dosage unit of 1 to 100 mg sumatriptan.

All the doses or dosage units of a physiologically acceptable salt of the migraine-active substances mentioned previously should be understood as being doses or dosages of the active compound itself.

Moreover a pharmaceutical composition according to the invention may be a kit of parts for the treatment or prevention of headache, migraine or cluster headaches, the kit comprising:

(a) a first enclosure containing a pharmaceutical composition comprising a therapeutically effective amount of the hydrochloride of the active substance base 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine (A) and one or more pharmaceutically acceptable diluents and/or carriers; and

(b) a second enclosure containing a pharmaceutical composition comprising sumatriptan or a physiologically acceptable salt thereof and one or more pharmaceutically acceptable diluents and/or carriers.

A third aspect of the present invention is the use of the hydrochloride of the active substance base 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine (A) in conjunction with the anti-migraine substance sumatriptan or a pharmaceutically acceptable salt thereof for preparing a pharmaceutical composition or a kit of parts for the simultaneous or sequential treatment or prevention of headache, migraine or cluster headaches.

Preferred embodiments thereof according to the invention as well as pharmaceutical compositions are mentioned above under the first and second aspects of the invention.

The above-mentioned anti-migraine substance sumatriptan is already on the market and is sold under the brand name Imigran®.

The hydrochloride of the active substance base (A) may be administered, for example, using the pharmaceutical formulations disclosed in WO 98/11128 or using one of the following pharmaceutical formulations:

capsules for powder inhalation containing 1 mg to 50 mg of active substance;

inhalable solution for atomisers containing 1 mg to 50 mg of active substance;

propellant-driven metered-dose aerosol containing 1 mg to 50 mg of active substance;

nasal spray containing 1 mg to 50 mg of active substance;

tablets containing 20 mg to 1000 mg of active substance;

capsules containing 20 mg to 1000 mg of active substance;

aqueous solution for nasal application containing 5 mg to 50 mg of active substance; or

suspension for nasal application containing 20 mg to 50 mg of active substance.

### EXPERIMENTAL SECTION

### EXAMPLE 1

The following experiments were carried out in order to investigate the pharmacological activity of the combination according to the invention:

Measurement of the Facial Skin Blood Flow

The facial skin blood flow was measured by a modified method as described by Escott et al. (Escott, K. J., Beattie, D. T., Connor, H. E., Brain, S. D. (1995), Trigeminal ganglion stimulation increases facial skin blood flow in the rat: a major role for calcitonin gene-related peptide, Brain Research, 669(1), 93-99). Fasting male Wistar rats (strain CHbb:THOM, 280-320 g) were anaesthetised with sodium pentobarbitone (initially with 60 mg/kg i.p. and maintained throughout the experiment with an intraperitoneal infusion of 30 mg/kg/h through a 23 G needle using a solution of 10 mg/ml). Both sides of the buccal region of the facial skin were shaved and depilated with a commercially available depilatory cream (Pilca, Schwarzkopf & Henkel, 40551 Düsseldorf, Germany). The trachea was fitted with a cannula, and the animals were artificially ventilated (80 breaths per minute) with ambient air enriched with oxygen. The body temperature was maintained at 37° C. using an automatic heating pad. The left femoral artery and left femoral vein were fitted with cannulas for continuous measurement of the arterial blood pressure or the intravenous administration of test compounds. A neuromuscular blockade was obtained by intravenous administration of pancuronium bromide (1 mg/kg/0.5 ml, 5 minutes before each electrical stimulation). The heart rate was derived from the blood pressure signal. The blood pressure and heart rate were recorded continuously during the course of the experiment in order to assess the level of anaesthesia and monitor the cardiovascular activities of the pharmaceutical compositions used in this study.

The animals were placed in a stereotactic frame, and a longitudinal cut was made in the scalp. A small hole was drilled in the skull (on the left or right) and a bipolar electrode (Rhodes SNEX-100, obtained from David Kopf Instruments, Tujunga, 91042 Calif., USA) was lowered by means of a micromanipulator into the trigeminal ganglion (0.32 cm dorsally of the bregma, ±0.30 cm laterally of the centre line and 0.95 cm below the dural surface). The position of the electrodes in the trigeminal ganglia was checked visually at the end of each experiment after the removal of the brain. The trigeminal ganglion was stimulated at 10 Hz, 1 mA, 1 msec for 30 seconds using a stimulator obtained from Hugo Sachs Elektronik (79232 March-Hugstetten, Germany). Microvascular changes in blood flow in the facial skin were measured by laser Doppler flow measurement using a Periflux Laser-Doppler system (PeriFLUX 4001, wavelength: 780 nm; time constant: 3 s; Perimed AB, Järfälla, S-17526, Sweden). Standard laser Doppler probes (PROBE 408) were arranged on each side of the face roughly 0.5 cm below the centre of the eye in an area innervated by the maxillary branch (V2) of the trigeminal nerve. Changes in blood flow were measured as flow in any desired units and expressed as the area under the flow curve (mm²) according to Escott et al. (1995).

### Experimental Procedure

After 30 minutes equilibration the animals were subjected to three periods of electrical stimulation separated by an interval of 30 minutes. The first stimulation was used as a control for the subsequent stimulations. Saline solution, individual compound and the combination were administered intravenously 5 minutes before the second stimulation.

The results are shown in Table 1 below. They show that the improved efficacy of the combination of the 5-HT_{1B/1D}-agonist sumatriptan with the hydrochloride of the CGRP antagonist (A) should result in a higher efficacy and allow smaller doses to be given, which should lead to similar efficacy with fewer side effects, and that the addition of two mechanisms possibly results in a reduced recurrence of headaches.

TABLE 1
Activity of the hydrochloride of the active substance base (A) in
conjunction with the anti-migraine substance sumatriptan against
migraine on the vasodilatation of the facial skin, induced by
electrical ganglion stimulation in the rat.

| | % of trigeminus | | % inhibition compared |
|---|---|---|---|
| treatment | stimulation | n | with the control value |
| saline solution (control) | 82.7 ± 4.4 | 11 | — |
| BIBN 4096 Cl | 60.3 ± 5.1 | 8 | 27.1 |
| (0.03 mg/kg) | | | |
| sumatriptan | 68.8 ± 6.8 | 7 | 16.8 |
| (1.0 mg/kg) | | | |
| BIBN 4096 Cl + | 26.6 ± 5.4^{a} | 6 | 67.8 |
| sumatriptan | | | |
| (0.03 mg + 1.0 mg)/kg | | | |

^{a}significant, p < 0.001, compared with sumatriptan

The following Examples describe pharmaceutical preparations which, unless otherwise, stated contain the free active substance base (A) as active substance

### EXAMPLE 2

Capsules for powder inhalation containing 1 mg of active substance

| composition: | |
|---|---|
| 1 capsule for powder | |
| inhalation contains: | |
| active substance | 1.0 mg |
| lactose | 20.0 mg |
| hard gelatine capsules | 50.0 mg |
| | 71.0 mg |

### Method of Preparation

The active substance is ground to the particle size required for inhalation. The ground active substance is homogeneously mixed with lactose and the mixture is packed into hard gelatine capsules.

### EXAMPLE 3

Inhalable solution for Respimat ® with 1 mg of active substance

| Composition: | | |
|---|---|---|
| 1 puff contains: | | |
| active substance | 1.0 | mg |
| benzalkonium chloride | 0.002 | mg |
| disodium edetate | 0.0075 | mg |
| purified water ad | 15.0 | μl |

### Method of Preparation

The active ingredient and benzalkonium chloride are dissolved in water and transferred into Respimat® cartridges.

### EXAMPLE 4

Inhalable solution for nebulisers containing 1 mg of active ingredient

| Composition: | | |
|---|---|---|
| 1 vial contains: | | |
| active ingredient | 0.1 | g |
| sodium chloride | 0.18 | g |
| benzalkonium chloride | 0.002 | g |
| purified water ad | 20.0 | ml |

### Method of Preparation

The active ingredient, sodium chloride and benzalkonium chloride are dissolved in water.

### EXAMPLE 5

Propellant gas-operated metered-dose aerosol containing 1 mg of active
substance

| Composition: | | |
|---|---|---|
| 1 puff contains: | | |
| active ingredient | 1.0 | mg |
| lecithin | 0.1% | |
| propellant gas ad | 50.0 | μl |

### Method of Preparation

The micronised active ingredient is homogeneously suspended in the mixture of lecithin and propellant gas. The suspension is transferred into a pressurised container with a metering valve.

### EXAMPLE 6

Nasal spray with 1 mg of active substance

| Composition: | | |
|---|---|---|
| 1 puff contains: | | |
| active substance | 1.0 | mg |
| mannitol | 5.0 | mg |
| disodium edetate | 0.05 | mg |
| ascorbic acid | 1.0 | mg |
| purified water ad | 0.1 | ml |

### Method of Preparation

The active ingredient and the excipients are dissolved in water and transferred into a suitable container.

### EXAMPLE 7

Injectable solution containing 5 mg of active substance per 5 ml

| Composition: | | |
|---|---|---|
| active substance in the form of the hydrochloride | 5 | mg |
| glucose | 250 | mg |
| human serum albumin | 10 | mg |
| glycofurol | 250 | mg |
| water for injections ad | 5 | ml |

### Preparation

Glycofurol and glucose are dissolved in water for injections (WfI). Human serum albumin is added and the active substance is dissolved with heating. The mixture is made up to the specified volume with WfI and transferred into ampoules under nitrogen gas.

### EXAMPLE 8

Injectable solution for subcutaneous administration
containing 5 mg of active substance per 1 ml

| Composition: | | |
|---|---|---|
| active substance | 5 | mg |
| glucose | 50 | mg |
| polysorbate 80 = Tween 80 | 2 | mg |
| water for injections ad | 1 | ml |

### Preparation

Glucose and polysorbate are dissolved in water for injections. The active substance is dissolved with heating or using ultrasound. The mixture is made up to the specified volume with WfI and transferred into ampoules under inert gas.

### EXAMPLE 9

Injectable solution containing 100 mg of active substance per 10 ml

| Composition: | | |
|---|---|---|
| active substance | 100 | mg |
| monopotassium dihydrogen phosphate = KH₂PO₄ | 12 | mg |
| disodium hydrogen phosphate = Na₂HPO₄.2H₂O | 2 | mg |
| sodium chloride | 180 | mg |
| human serum albumin | 50 | mg |
| Polysorbate 80 | 20 | mg |
| water for injections ad | 10 | ml |

### Preparation

Polysorbate 80, sodium chloride, monopotassium dihydrogen phosphate and disodium hydrogen phosphate are dissolved in water for injections (WfI). Human serum albumin is added. The active substance is dissolved with heating. The mixture is made up to the specified volume with WfI and transferred into ampoules.

### EXAMPLE 10

Lyophilisate containing 10 mg of active substance

| Composition: | | |
|---|---|---|
| Active substance in the form of the hydrochloride | 10 | mg |
| Mannitol | 300 | mg |
| water for injections ad | 2 | ml |

### Preparation

Mannitol is dissolved in water for injections (WfI) and the active substance is added with heating. The preparation is made up to the specified volume with WfI, transferred into vials and freeze-dried.

| Solvent for lyophilisate: | |
|---|---|
| Polysorbate 80 = Tween 80 | 20 mg |
| mannitol | 200 mg |
| water for injections ad | 10 ml |

### Preparation

Polysorbate 80 and mannitol are dissolved in water for injections (WfI) and transferred into ampoules.

### EXAMPLE 11

Lyophilisate containing 5 mg of active substance

| Composition: | | |
|---|---|---|
| active substance | 5 | mg |
| polar or nonpolar solvent (which can be | ad 1 | ml |
| removed by freeze-drying) | | |

### Preparation

The active substance is dissolved in a suitable solvent, transferred into vials and freeze-dried.

| Solvent for lyophilisate: | | |
|---|---|---|
| Polysorbate 80 = Tween 80 | 5 | mg |
| Mannitol | 100 | mg |
| water for injections | ad 2 | ml |

### Preparation

Polysorbate 80 and mannitol are dissolved in water for injections (WfI) and transferred into ampoules.

### EXAMPLE 12

Tablets containing 20 mg of active substance

| Composition: | |
|---|---|
| active substance | 20 mg |
| lactose | 120 mg |
| maize starch | 40 mg |
| magnesium stearate | 2 mg |
| Povidone K25 | 18 mg |

### Preparation

The active substance, lactose and maize starch are homogeneously mixed, and granulated with an aqueous solution of Povidone, mixed with magnesium stearate and compressed in a tablet press. Weight of tablet: 200 mg.

### EXAMPLE 13

Capsules containing 20 mg of active substance

| Composition: | |
|---|---|
| active substance | 20 mg |
| maize starch | 80 mg |
| highly dispersed silica | 5 mg |
| magnesium stearate | 2.5 mg |

### Preparation

The active substance, maize starch and silica are homogeneously mixed and mixed with magnesium stearate. The mixture is packed into 8 size 3 hard gelatine capsules in a capsule filling machine.

### EXAMPLE 14

Suppositories containing 50 mg

| Composition: | |
|---|---|
| active substance | 50 mg |
| hard fat (Adeps solidus) q.s. ad | 1700 mg |

### Preparation

The hard fat is melted at about 38° C. The ground active substance is homogeneously dispersed in the molten hard fat and after cooling to about 35° C. it is poured into chilled moulds.

### EXAMPLE 15

Aqueous solution for nasal administration containing 10 mg of active
substance

| Composition: | |
|---|---|
| active substance in the form of the | 10.0 mg |
| hydrochloride | |
| methylparahydroxybenzoate (PHB) | 0.01 mg |
| propylparahydroxybenzoate (PHB) | 0.005 mg |
| purified water ad | 1.0 ml |

### Preparation

The active substance is dissolved in purified water and methyl- and propyl-PHB are added. The solution is made up to the specified volume with purified water, filtered sterile and transferred into a suitable container.

### EXAMPLE 16

Aqueous solution for nasal administration containing 5 mg
of active substance

| Composition: | |
|---|---|
| active substance | 5 mg |
| 1,2-propanediol | 300 mg |
| hydroxyethylcellulose | 5 mg |
| sorbic acid | 1 mg |
| purified water ad | 1 ml |

### Preparation

The active substance is dissolved in 1,2-propanediol. A hydroxyethylcellulose solution in purified water containing sorbic acid is prepared and added to the solution of active substance. The solution is filtered sterile and transferred into a suitable container.

### EXAMPLE 17

Aqueous solution for intravenous administration containing 5 mg
of active substance

| Composition: | |
|---|---|
| active substance | 5 mg |
| 1,2-propanediol | 300 mg |
| mannitol | 50 mg |
| water for injection (Wfl) ad | 1 ml |

### Preparation

The active substance is dissolved in 1,2-propandiol and the solution is made up to roughly the specified volume with WfI. The mannitol is added, and the preparation is made up to roughly the specified volume with WfI. The solution is filtered sterile, transferred into individual containers and autoclaved.

### EXAMPLE 18

Liposomal formulation for intravenous injection containing 7.5 mg
of active substance

| Composition: | |
|---|---|
| active substance | 7.5 mg |
| egg lecithin, e.g. Lipoid E 80 | 100.0 mg |
| cholesterol | 50.0 mg |
| glycerol | 50.0 mg |
| water for injections ad | 1.0 ml |

### Preparation

The active substance is dissolved in a mixture of lecithin and cholesterol. The solution is added to a mixture of glycerol and WfI and homogenised by high pressure homogenisation or using the Microfluidizer technique. The liposome formulation obtained is transferred into a suitable container under aseptic conditions.

### EXAMPLE 19

Suspension for nasal administration containing 20 mg of
active substance

| Composition: | | |
|---|---|---|
| active substance | 20.0 | mg |
| carboxymethylcellulose (CMC) | 20.0 | mg |
| sodium monohydrogen phosphate/ | q.s. | |
| sodium dihydrogen phosphate buffer | | |
| pH 6.8 | | |
| sodium chloride | 8.0 | mg |
| methylparahydroxybenzoate | 0.01 | mg |
| propylparahydroxybenzoate | 0.003 | mg |
| purified water ad | 1.0 | ml |

### Preparation

The active substance is suspended in an aqueous CMC solution; the other ingredients are added to the suspension one after the other and the suspension is made up to the specified volume with purified water.

### EXAMPLE 20

Aqueous solution for subcutaneous administration containing
10 mg of active substance

| Composition: | | |
|---|---|---|
| active substance | 10.0 | mg |
| sodium monohydrogen phosphate/ | 7.0 | |
| sodium dihydrogen phosphate buffer | | |
| q.s. ad pH | | |
| sodium chloride | 4.0 | mg |
| water for injections ad | 0.5 | ml |

### Preparation

The active substance is dissolved in the phosphate buffer solution, and after the addition of the common salt the solution is made up to the specified volume with water. The solution is filtered sterile, transferred into a suitable container and autoclaved.

### EXAMPLE 21

Aqueous solution for subcutaneous administration containing
5 mg of active substance

| Composition: | | |
|---|---|---|
| active substance | 5.0 | mg |
| polysorbate 80 | 0.5 | mg |
| water for injections | 0.5 | ml |

### Preparation

The active substance is suspended in the polysorbate 80 solution and comminuted to a particle size of about 1 μm using a suitable dispersing technique (e.g. wet grinding, high pressure homogenisation, microfluidisation and the like). The suspension is transferred into a corresponding container under aseptic conditions.

We claim:

1. A method for treating or preventing a condition selected from headaches and migraine, said method comprising the simultaneous or sequential administration of: (1) a therapeutically effective amount of the hydrochloride of 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine and (2) a therapeutically effective amount of sumatriptan, or one of the physiologically acceptable salts thereof, to a person in need of such treatment.

2. A method according to claim 1, wherein the condition is a cluster headache.

3. A method according to claim 1, wherein the sequential administration comprises first administering a therapeutically effective amount of the hydrochloride of 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine and subsequently administering a therapeutically effective amount of sumatriptan, or one of the physiologically acceptable salts thereof.

4. A method according to claim 1, wherein the sequential administration comprises first administering a therapeutically effective amount of sumatriptan, or one of the physiologically acceptable salts thereof, and subsequently administering a therapeutically effective amount of the hydrochloride of 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine.

5. A method according to claim 1, wherein the hydrochloride of 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine is administered by intravenous or subcutaneous route in a dosage of 0.0001 to 3 mg/kg of body weight or by oral, nasal, rectal or inhalative route in a dosage of 0.1 to 10 mg/kg of body weight, once, twice or three times a day, and sumatriptan or a physiologically acceptable salt thereof is administered:
by oral route in a dosage of 0.14 to 1.5 mg/kg of body weight once, twice or three times a day, or
by intravenous or subcutaneous route in a dosage of 0.009 to 0.1 mg/kg of body weight once or twice a day, or
by rectal route in a dosage of 0.04 to 0.36 mg/kg of body weight once or twice a day, or
by inhalation in a dosage of 0.057 to 0.57 mg/kg of body weight once or twice a day, or
by nasal route in a dosage of 0.036 to 0.29 mg/kg of body weight once or twice a day.

6. A pharmaceutical composition comprising a therapeutically effective amount of a combination of the hydrochloride of 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine and sumatriptan or one of the physiologically acceptable salts thereof.

7. A pharmaceutical composition according to claim 6, comprising a single dosage unit of 0.1 to 10 mg of the hydrochloride of 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine and a single dosage unit of 1 to 100 mg sumatriptan.

8. A kit comprising:
(a) a first enclosure containing a pharmaceutical composition comprising a therapeutically effective amount of the hydrochloride of 1-[N²-[3,5-dibromo-N-[[4-(3,4-dihydro-2(1H)-oxoquinazolin-3-yl)-1-piperidinyl]-carbonyl]-D-tyrosyl]-L-lysyl]-4-(4-pyridinyl)-piperazine and one or more pharmaceutically acceptable diluents and/or carriers; and
(b) a second enclosure containing a pharmaceutical composition comprising sumatriptan, or a physiologically acceptable salt thereof, and one or more pharmaceutically acceptable diluents and/or carriers.
